(19) United States
(12) Patent Application Publication
(10) Pub. No.: US 2012/0135056 A1
(43) Pub. Date: May 31, 2012

(54) ANTIPERSPIRANT COMPOSITIONS AND PRODUCTS HAVING PREDETERMINED EFFECTS AND METHODS FOR MAKING THE SAME

(75) Inventors: Travis T. Yarlagadda, Phoenix, AZ (US); Thomas Doering, Scottsdale, AZ (US)

(73) Assignee: The Dial Corporation, Scottsdale, AZ (US)

(21) Appl. No.: 12/956,142

(22) Filed: Nov. 30, 2010

(51) Int. Cl. A61K 8/96; A61Q 15/00; B29C 39/12; A61K 8/02

(52) U.S. Cl. 424/401; 424/65; 424/66; 264/259

(57) ABSTRACT

A personal care product comprises an antiperspirant product housed within a container. The antiperspirant product comprises a first portion comprising a first ingredient. A second portion is macroscopically separated from the first portion. The second portion has a composition different from the first portion and comprises a second ingredient effective to interact with the first ingredient to produce a predetermined effect.

### FIELD OF THE INVENTION

The present invention generally relates to personal care antiperspirant compositions, products and methods for making the same, and more particularly relates to antiperspirant compositions that exhibit antiperspirant efficacy and other desirable effects, antiperspirant products comprising such antiperspirant compositions, and methods for manufacturing such antiperspirant compositions and products.

### BACKGROUND OF THE INVENTION

Antiperspirants and deodorants are well known personal care products used to prevent or eliminate sweat and body odor caused by sweat. The compositions come in a variety of forms and may be formulated, for example, into aerosols, pumps, sprays, liquids, roll-ons, lotions, creams, and sticks (both hard and soft), etc.

There are various types of stick antiperspirant compositions that are desirable by large majority of the population because of their ease of application and the presence of active antiperspirant compounds, e.g. antiperspirant salts, which prevent or block the secretion of sweat and its accompanying odors. In one type, an antiperspirant salt is suspended in an anhydrous vehicle often including a solid water-insoluble wax. In a second type, an antiperspirant salt is dissolved in a liquid vehicle such as propylene glycol and gelled with a gelling agent such as dibenzylidene sorbitol. A third type includes an emulsion of an aqueous phase containing the antiperspirant salt and an oil phase containing, for example, a volatile silicone, fragrances, gellants, and other additives.

Stick antiperspirant products include an antiperspirant composition within a container. During use of the product, the top of the container is removed and the application surface of the composition is contacted with the underarm by swiping or rubbing the stick across the skin. Sometimes the product also includes an undercap, or factory seal, covering the application surface that is removed prior to first use. During use, some of the composition is transferred to the skin, and a container generally also includes some mechanism for moving the composition upwards through the container to continue to provide an exposed application surface.

Commercial markets for antiperspirant and deodorant products are highly competitive with consumers wanting increased antiperspirant efficacy and various other effects from these products. One problem for antiperspirant and deodorant manufacturers is that the ingredients and/or additives used in the product for antiperspirant efficacy and various other effects may comprise two or more different ingredients and/or additives that when combined during manufacturing interact with each other, gradually reducing, for example, antiperspirant efficacy and/or other desirable effects. This may result in the antiperspirant product having a limited shelf life, becoming less effective and/or losing other desirable effects over time. Another problem is that such antiperspirant or deodorant compositions may not provide efficacy and other desirable effects when the user needs it most throughout the day.

Accordingly, it is desirable to provide antiperspirant products that exhibit strong antiperspirant efficacy and other various effects which have good shelf life and/or are more responsive to the user when needed most. In addition, it is desirable to provide methods for manufacturing such antiperspirant products. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Antiperspirant products and methods for manufacturing antiperspirant products are provided herein. In accordance with an exemplary embodiment, a personal care product is provided. The personal care product comprises a container. An antiperspirant product is housed within the container and comprises a first portion comprising a first ingredient. A second portion is macroscopically separated from the first portion. The second portion has a composition different from the first portion and comprises a second ingredient effective to interact with the first ingredient to produce a predetermined effect.

In accordance with another exemplary embodiment, a method for manufacturing an antiperspirant product is provided. The method comprises the steps of depositing a first portion of the antiperspirant product into a mold and allowing the first portion to at least partially solidify. The first portion comprises a first ingredient. A second portion of antiperspirant product is deposited into a mold and is allowed to at least partially solidify. The second portion has a composition different from the first portion and comprises a second ingredient effective to interact with the first ingredient to produce a predetermined effect.

### BRIEF DESCRIPTION OF THE DRAWINGS

The present invention will hereinafter be described in conjunction with the following drawing figures, wherein like numerals denote like elements, and wherein:

FIG. 1 is a perspective view of an antiperspirant product in accordance with an exemplary embodiment;

FIG. 2 is a top view of the antiperspirant product of FIG. 1;

FIG. 3 is an exploded perspective view of an antiperspirant composition comprising the antiperspirant product of FIG. 1 in a container in accordance with an exemplary embodiment;

FIG. 4 is a perspective view showing the initial step of a filling assembly being inserted into a container in a process for manufacturing the antiperspirant composition of FIG. 3 in accordance with an exemplary embodiment;

FIG. 5 is a perspective view of an outer nozzle assembly of the filling apparatus shown in FIG. 4;

FIGS. 6 and 7 are cross-sectional views of the center nozzle tube shown in FIG. 5;

FIG. 8 is an enlarged detailed view of a portion of the outer nozzle assembly of FIG. 5;

FIG. 9 is a perspective view of an inner nozzle assembly of the filling apparatus shown in FIG. 4;

FIG. 10 is a perspective view of the inner nozzle assembly shown in FIG. 9, with one half of the nozzle housing removed to show the interior of the inner nozzle assembly;

FIG. 11 is an elevational view of the inner nozzle assembly shown in FIG. 10 and

FIG. 12 is an exploded view showing internal components of the inner nozzle assembly;

FIG. 13 is a perspective view showing a further step in the manufacturing process of FIG. 4;

FIG. 14 is a cross-sectional view taken along line 14-14 in FIG. 13;

FIG. 15 is a perspective view showing a further step in the manufacturing process of FIGS. 4 and 13;

FIG. 16 is a cross-sectional view taken along line 16-16 in FIG. 15;

FIG. 17 is a perspective view showing a further step in the manufacturing process of FIGS. 4, 13, and 15;

FIG. 18 is a cross-sectional view taken along line 18-18 in FIG. 17;

FIG. 19 is a partially exploded perspective view of a portion of a nozzle assembly used in an alternative process for manufacturing the antiperspirant composition illustrated in FIG. 3 in accordance with an exemplary embodiment;

FIG. 20 is a fully exploded perspective view of the nozzle assembly of FIG. 19;

FIG. 21 is a cross sectional view showing the nozzle assembly of FIG. 19 filling a container;

FIG. 22 is an exploded perspective view of the nozzle assembly used in another alternative process for manufacturing the antiperspirant composition illustrated in FIG. 3 in accordance with an exemplary embodiment; and

FIG. 23 is a flowchart of an example of a process for manufacturing an antiperspirant composition and product in accordance with the present invention.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The various embodiments contemplated herein relate to antiperspirant products that exhibit antiperspirant efficacy and other predetermined effects to a user upon application and/or upon perspiration. Some non-limiting examples of the predetermined effects are enhanced wetness protection, antibacterial protection, enhanced fragrance, and reduced staining by color additives in the antiperspirant product so that the color additives do not transfer to the user's cloths for example. The antiperspirant product comprises two distinct portions, macroscopically separated from each other, each having a different composition. One or both portions may have an active antiperspirant ingredient or ingredients but a first portion has a first ingredient dispersed throughout that portion, while a second different ingredient is dispersed throughout the second portion. When the antiperspirant product is applied to an underarm, for example, the two portions of the antiperspirant product are intermingled or mixed together. The first ingredient and the second ingredient are effective to interact, e.g., chemically or physically, with each other to produce a predetermined effect. In one exemplary embodiment, the first and second ingredients interact with each other upon the intermingling of the first and second portions during application to the user's underarm to provide, for example, antibacterial protection, enhanced fragrance, or staining protection from the color additives in the antiperspirant product. In another exemplary embodiment, the first and/or second ingredients are captured on a plurality of silica particles that are disposed throughout their corresponding portion. When the antiperspirant product is applied to the user's underarm, the first and second portions intermingle, however, the first and/or second ingredients remain separated and captured on their respective silica particles. Preferably, the silica particles are porous and hydrophilic such that when the user perspires, the perspiration is readily absorbed by the silica particles, displacing or driving out the first and/or second ingredients from the silica particles so that the first and second ingredients are free to interact with each other. The inventors have unexpectedly found that by keeping the first and second ingredients macroscopically separated from each other in their respective portions prior to application and further, by allowing the ingredients to intermingle either upon application or upon contact with the user's perspiration, the antiperspirant product has an excellent shelf-life and provides the user a desirable effect throughout the day when it is most needed e.g., when the user perspires. Without being macroscopic separation, the premature interaction of the first and second ingredients during manufacturing would result in loss of the predetermined effects as well as loss of overall antiperspirant efficacy and product aesthetics.

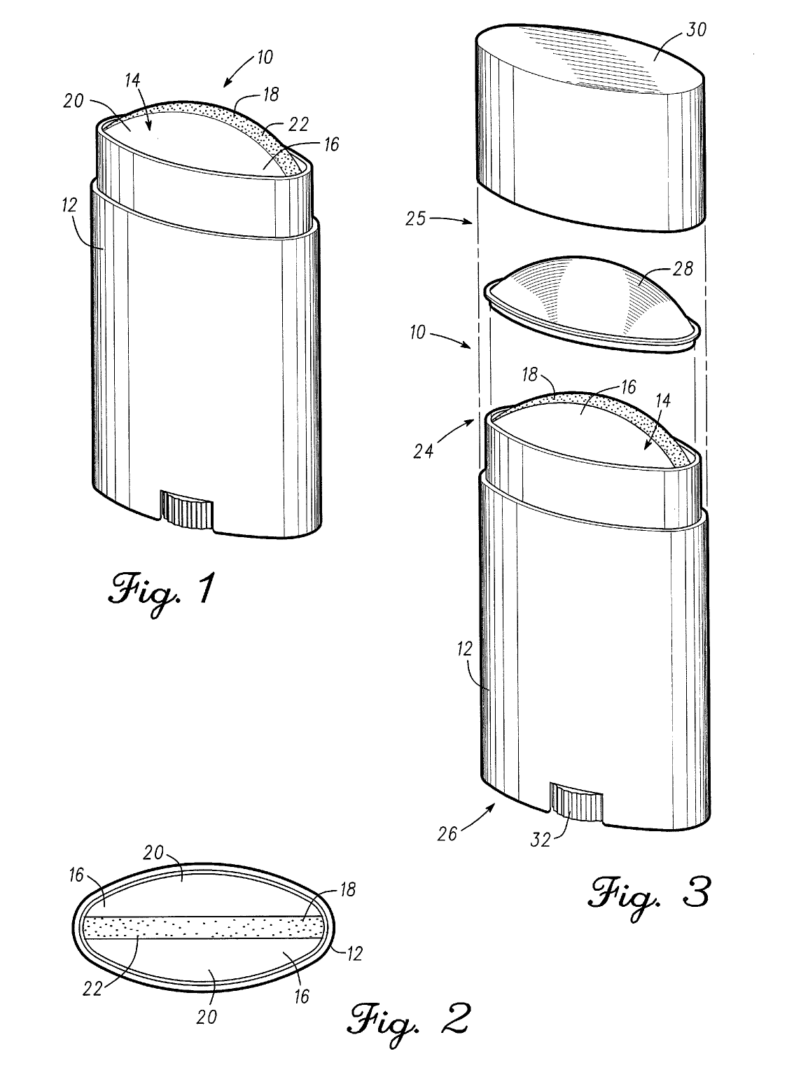

Referring to FIGS. 1 and 2, a personal care antiperspirant product 10 in accordance with an exemplary embodiment comprises a first portion 16 and a second portion 18. The term “portion” as used herein, includes the section or sections of the antiperspirant product having the same composition, for example, two sections having the same composition but separated by a third section (for example, a central stripe) having a different composition constitute a single “portion.” The first portion 16 may have a color different from that of second portion 18 or the portions may be of the same color. As shown, the first and second portions 16 and 18 are visibly and/or macroscopically separated in non-mutual spatial relationship with each other. The antiperspirant product 10 has an application surface 14 that is substantially dome-shaped and that is configured to be applied to skin, such as, for example, an underarm. The antiperspirant product 10 also may comprise a container or dispenser 12 for dispensing first portion and second portion 16 and 18 to the skin.

In one exemplary embodiment, the first portion 16 is an outer portion and the second portion 18 is an inner portion and the application surface 14 comprises a surface 20 of first portion 16 that is bisected by an adjacent surface 22 of the second portion 18. The first portion 16 and the second portion 18 may both be solid wax formulations. Alternatively, one of the first and second portions 16 or 18 may be a solid wax formulation while the other of the first and second portions 16 or 18 is an emulsion formulation. As illustrated, the first portion 16 is positioned on outside regions of the antiperspirant product 10 while the second portion 18 is positioned between the regions of the first portion 16. This configuration is a preferred configuration because the first portion 16 has a melting point that is higher than the melting point of the second portion 18. During manufacture, as described in more detail below, the second portion 18 is formed after the first portion 16 and thus, will not melt the already prepared first portion 16. However, it will be appreciated that the invention is not limited to the configuration of the first portion 16 and the second portion 18 illustrated in FIGS. 1 and 2. Rather, it will be appreciated that the second portion 18 can be the outer portion and the first portion 16 can be the inner portion.

It will be appreciated that the first portion 16 and the second portion 18 also may have other configurations. For example, the second portion 18 may be completely surrounded by the first portion 16 or vice versa. Alternatively, rather than forming one strip bisecting the first portion 16, the second portion 18 may form two or more strips. The antiperspirant product 10 may also comprise a third portion and other additional portions that do not comprise the compositions of the first portion 16 and the second portion 18. The first portion 16 and the second portion 18 may take any other configuration suitable for applying the portions to skin. The surface 20 of the first portion 16 and the surface 22 of the second portion 18 each comprises at least 15%, and preferably at least 25%, of the application surface 14. Each of the surfaces 20 and 22 may even comprise, for example, at least 40% of the application surface 14.

The first and second portions 16 and 18 are made of formulations each having different compositions than the other. The first portion 16 contains a first ingredient and the second portion 18 contained a second ingredient that is different than the first ingredient and is effective to interact with the first ingredient to produce a predetermined effect. In an exemplary embodiment, the first ingredient is dispersed throughout the first portion 16 and not the second portion 18, and the second ingredient is dispersed throughout the second portion 18 and not the first portion 16. Alternatively, the first ingredient may be dispersed throughout both the first and second portions 16 and 18, while the second ingredient is dispersed throughout the second portion 18 and not the first portion 16. In yet another scenario, the first ingredient may be dispersed throughout the first portion 16 and not the second portion 18, while the second ingredient is dispersed throughout both the first and second portions 16 and 18.

In an exemplary embodiment, the first ingredient is an active antiperspirant salt, the second ingredient is a release enhancer and the predetermined effect is enhanced wetness protection. Active antiperspirant salts contain at least one active ingredient, typically metal salts, that are thought to reduce sweating by diffusing through the sweat ducts of apocrine glands (sweat glands responsible for body odor) and hydrolyzing in the sweat ducts, where they combine with proteins to form an amorphous metal hydroxide agglomerate, plugging the sweat ducts so sweat can not diffuse to the skin surface. Suitable active antiperspirant salts for interacting with the release agent comprise aluminum zirconium pentachlorohydrex GLY, aluminum zirconium trichlorohydrex GLY, aluminum chlorohydrate, or mixtures thereof. Suitable release enhancers for interacting with the active antiperspirant salt comprise alkoxylated compounds, ethoxylated compounds, PEG-25 propylene glycol stearate, ceteareth-12, ceteareth-20, ceteareth-30, PPG-3 benzyl ether ethylhexanoate, organofunctional siloxane, glyceral stearate, PEG-100 stearate, C20-40 pareth-10, phospholipids, linoleamidopropyl PG-dimonium chloride phosphate dimethicone, hydrogenated lecithin, unhydrogenated lecithin, phosphatidylcholine, phosphatidylethanolamine, phosphatidylerine, phosphatidylglycerol, cardiolipin, or mixtures thereof. Other suitable active antiperspirant salts and/or release enhancers known to those skilled in the art may also be used.

Preferably, the first portion 16 comprises the active antiperspirant salt in an amount of from about 10 to about 50 weight percent (USP), and more preferably of from about 15 to about 30 weight percent (USP), and the second portion 18 comprises the release enhancer in an amount of from about 0.1 to about 24 weight percent (wt. %), and more preferably of from about 0.5 to about 6 wt. %. As used herein, weight percent (USP) or wt. % (USP) of an antiperspirant salt is calculated as anhydrous weight percent in accordance with the U.S.P. method, as is known in the art. This calculation excludes any bound water and glycine.

In another exemplary embodiment, the first ingredient is an antibacterial agent, the second ingredient is a solubilizer and the predetermined effect is antibacterial protection. The solubilizer facilitates solubilizing of the antibacterial agent in body generated moisture where the antibacterial agent can readily kill the bacteria responsible for malodor and/or inhibit or suppress or interfere with the bacterial enzymatic pathway that produces malodor. Suitable antibacterial agents for interacting with the solubilizer comprise triclocarban, triclosan, benzalconium chloride, phenoxy ethanol, chlorohexidine, or mixtures thereof. Suitable solubilizers for interacting with the antibacterial agent comprise polyethylene glycols, polypropylene glycols, polyglycols, or mixtures thereof. Other suitable antibacterial agent and/or solubilizers known to those skilled in the art may also be used.

Preferably the first portion 16 comprises the antibacterial agent in an amount of from about 0.1 to about 6 wt. %, and more preferably of from about 0.3 to about 4 wt. %. The second portion 18 preferably comprises the solubilizer in an amount of from about 0.3 to about 14 wt. %, and more preferably of from about 0.5 to about 5 wt. %.

In one exemplary embodiment, the first ingredient is a fragrance extract, the second ingredient is a solvent and the predetermined effect is enhanced or relatively longer lasting fragrance. Suitable fragrance extracts for interacting with the solvent comprise absolutes, crystalline, resinoids, resins, waxes, or mixtures thereof. Absolutes are natural fragrance materials which are extracted from various plant parts. Crystalline are natural fragrance extracts having a high percentage of crystalline fragrance substances that can be readily dissolved in perfume oil. Resinoids are natural extracts from resins or plant parts that contain essential oils. Resins are primarily solid or semi-solid organic plant secretions. Waxes are natural extracts consisting of hydrocarbons or esters of fatty acids that are generally insoluble in water but are soluble in many organic solvents. Other suitable fragrance extracts known to those skilled in the art may also be used.

Preferably, the solvent for interacting with the fragrance extract is ethanol, however, other suitable solvents for dissolving fragrance extracts known to those skilled in the art may also be used. Preferably, the first portion 16 comprises the fragrance extract in an amount of from about 0.1 to about 6 wt. %, and more preferably of from about 0.3 to about 3 wt. %. The second portion 18 preferably comprises the solvent in an amount of from about 0.3 to about 12 wt. %, and more preferably of from about 0.5 to about 5 wt. %.

In another exemplary embodiment, the first ingredient is a dye transfer inhibitor, the second ingredient is a color additive and the predetermined effect is reduced staining by the color additive. Suitable dye transfer inhibitors for interacting with the color additive comprise poly(N-vinylpyrrolidone), poly(vinylpyridine N-oxide), or mixtures thereof. Suitable color additives for interacting with the dye transfer inhibitor comprise dyes, pigments, and mixtures thereof. Several examples of suitable dyes and/or pigments for color additives can be found at the follow site: http://www.fda.gov/ForIndustry/ColorAdditives/ColorAdditiveInventories/ucm115641htm#f tnote6. Other suitable color additives and/or dye transfer inhibitors known to those skilled in the art may also be used.

Preferably, the first portion 16 comprises the dye transfer inhibitor in an amount of from about 0.1 to about 10 wt. %, and more preferably of from about 0.3 to about 5 wt. %. The second portion 18 preferably comprises the color additive in an amount of from about 0.01 to about 3 wt. %.

In an exemplary embodiment, the first ingredient is an active antiperspirant salt, the second ingredient is an activity enhancement agent and the predetermined effect is enhanced wetness protection. As discussed above, suitable active antiperspirant salts for interacting with an activity enhancement agent comprise aluminum zirconium pentachlorohydrex GLY, aluminum zirconium trichlorohydrex GLY, aluminum chlorohydrate, or mixtures thereof. Suitable activity enhancement agents for interacting with the active antiperspirant salt comprise calcium chloride, glycine, or mixtures thereof. Other suitable active antiperspirant salts and/or activity enhancement agents known to those skilled in the art may also be used.

Preferably, the first portion 16 comprises the active antiperspirant salt in an amount of from about 15 to about 30 wt. % (USP), and more preferably of from about 15 to about 20 wt. % (USP). The second portion 18 preferably comprises the activity enhancement agent in an amount of from about 0.3 to about 12 wt. %, and more preferably of from about 0.5 to about 6 wt. %.

In one exemplary embodiment, the first ingredient is captured or adhered to the surfaces of a first plurality of silica particles that are in the form of a solid powder that is dispersed throughout the first portion 16. As such, the first ingredient is effectively dispersed throughout the first portion 16. Preferably, the silica particles are porous and hydrophilic. In an exemplary embodiment, the silica particles are hydrophilic forms of precipitated silica having a BET surface area of from about 50 to about 1000 m²/g, preferably from about 100 to about 700 m²/g, and most preferably from about 150 to about 600 m²/g; a particle size d₅₀, determined by laser diffraction, from about 2 to about 130 μm, preferably from about 3 to about 20 μm; a DOA (dioctyl acetate) absorption from about 150 to about 400 g DBP/100 g silica, preferably from about 200 to about 350 g DBP/100 g silica. In one example, the silica particles are precipitated silica particles. One such type is Sipernat® 22S, which is manufactured and sold by Evonik Industries of Mobile, Ala. and is comprised of fine silica particles with high absorption capacity for liquids, especially water. The first ingredient is adsorbed onto, absorbed into and effectively captured by the porous hydrophilic silica particles. When the porous hydrophilic silica particles are exposed to an amount of moisture, e.g. perspiration, the moisture is readily taken up by the porous hydrophilic silica particles displacing and releasing (e.g. eluting) the first ingredient from the particles for interacting with the second ingredient. Preferably, the first portion 16 comprises the first plurality of silica particles in an amount of from about 1.0 to about 4.0 wt. %. Other suitable silica particles or hydrophilic carriers known to those skilled in the art may also be used to disperse the cooling sensation agent throughout the first portion 16.

In another exemplary embodiment, the second ingredient is captured or adhered to the surfaces of a second plurality of silica particles (silica particles as described above) that are in the form of a solid powder and that are preferably porous and hydrophilic. Thus, the second ingredient is effectively dispersed throughout the second portion 18 as particles of the solid powder. In one example, the second ingredient is adsorbed onto, absorbed into and effectively captured by the porous hydrophilic silica particles. When the porous hydrophilic silica particles are exposed to an amount of moisture, e.g. perspiration, the moisture is readily taken up by the porous hydrophilic silica particles, displacing and releasing (e.g. eluting) the second ingredient from the particles for interacting with the first ingredient. Preferably, the second portion 18 comprises the second plurality of silica particles in an amount of from about 1.0 to about 4.0 wt. %.

Additionally, one or both of the first and second portions 16 and 18 may contain a relatively low amount of an unbound first and/or second ingredient that is not captured on the silica particles. The unbound first ingredient intermingles with the unbound second ingredient to provide the predetermined effect when the antiperspirant product is initially applied to the user and for some period throughout the day thereafter.

In one exemplary embodiment, the first ingredient and/or the second ingredient are microencapsulated into rupturable membranes, which preferably rupture during frictional application of the antiperspirant product to the user's underarm. In a non-limiting example, the first portion 16 is a solid wax formulation containing a plurality of microencapsulated hydrophobic antibacterial agent, and the second portion 18 is also a solid wax formulation that contains a plurality of microencapsulated solubilizer. In another non-limiting example, the first portion 16 is an emulsion formulation comprising a water phase and an oil phase. In the oil phase, a plurality of microencapsulated activity enhancing agent is dispersed throughout. The second portion 18 is a solid wax formulation containing an active aluminum salt. Because the activity enhancing agent is microencapsulated in the oil phase of the first portion 16, additional active aluminum salts may also be added to the water phase of the first portion 16 without interacting with the activity enhancing agent until the antiperspirant product is applied and the membranes rupture. Suitable rupturable membranes for microencapsulating include but are not limited to starch membranes and carboxylate membranes. Other suitable membranes for microencapsulating antiperspirant ingredients known to those skilled in the art may also be used.

In an exemplary embodiment, one of the first and second portions 16 or 18 is an emulsion portion (discussed in further detail below), and the other of the first and second portions 16 or 18 is a solid wax portion. The emulsion portion is a water-in-oil emulsion comprising a water phase mixed with an oil phase. Preferably, the emulsion portion comprises a water phase in an amount of about 35 to about 45 weight percent (wt. %) of the total emulsion portion and an oil phase in an amount of about 55 to about 65 wt. % of the emulsion portion. The emulsion portion preferably has a soft, non-flowing, solid composition that can be rubbed or wiped across the skin, particularly the underarm. However, the various embodiments are not so limited and the emulsion portion can also have a gel, cream, or lotion consistency. The solid composition may be substantially snow white in color, thus suggesting a clean and/or sterile nature.

In one exemplary embodiment, the water phase of the emulsion portion comprises a water-soluble active antiperspirant compound. Some active antiperspirant compounds that may be used in the emulsion portion include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₙCl_{b}x(H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐx(H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the antiperspirant compound is aluminum sesquichlorohydrate (anhydrous) with glycine and calcium chloride. In another embodiment, the emulsion portion comprises an active antiperspirant compound present in the amount of 0 to about 25 wt. % (USP). In a more preferred embodiment, the antiperspirant compound comprises aluminum sesquichlorohydrate (anhydrous) and glycine and calcium chloride at an active level of about 12 to about 25 wt. % (USP).

In an exemplary embodiment, the water phase also comprises at least one water soluble carrier/solubilizer present in a sufficient amount to solubilize or disperse the water phase ingredients of the antiperspirant product. Such carriers/solubilizers suitable for use in the antiperspirant product include, but are not limited to, propylene glycol, glycerol, dipropyl glycol, ethylene glycol, butylene glycol, propylene carbonate, dimethyl isosorbide, hexylene glycol, ethanol, n-butyl alcohol, n-propyl alcohol, isopropyl alcohol, and the like. In a preferred embodiment, the water phase comprises propylene glycol and, in a more preferred embodiment, the water phase comprises propylene glycol in an amount of about 4 to about 12 wt. % of the total first portion. In addition to the carrier/solubilizer, the water phase comprises water. The water evaporates from the antiperspirant product upon application of the antiperspirant product to the skin, providing a cooling sensation to the skin.

The water phase also may comprise optional ingredients that serve a particular purpose. In one exemplary embodiment, the water phase comprises an activator for the active antiperspirant compound. In another embodiment, the water phase comprises calcium chloride in an amount of about 0.7 to about 2 wt. % of the total emulsion portion.

The oil phase of the emulsion portion comprises an emulsifier of cetyl PEG/PPG-10/1 dimethicone, in accordance with an exemplary embodiment. Cetyl PEG/PPG-10/1 dimethicone is a copolymer of cetyl dimethicone and an alkoxylated derivative of dimethicone containing an average of 10 moles of ethylene oxide and 1 mole of propylene oxide. The use of cetyl PEG/PPG-10/1 dimethicone as an emulsifier in the emulsion portion causes the emulsion portion to exhibit skin feel characteristics that are typical of deodorant products. For example, with cetyl PEG/PPG-10/1 dimethicone, the antiperspirant products glide onto the skin with less friction while still maintaining a solid consistency for easy application. In addition, when applied, the antiperspirant products are smoother than typical antiperspirants and thus give the skin a smoother and softer feel. In a preferred embodiment, the oil phase comprises cetyl PEG/PPG-10/1 dimethicone in an amount of about 1 to about 4 wt. % of the total antiperspirant product.

Further included in the oil phase of the emulsion portion is at least one structurant and/or gellant (hereinafter referred collectively as structurant) that facilitates the solid consistency of the emulsion portion. Naturally-occurring or synthetic waxy materials or combinations thereof can be used as such structurants. Examples of these waxy materials include those fatty alcohols that are solid at room temperature and hydrocarbon waxes or silicone waxes. Such materials are widely available, and by suitable selection of the materials themselves and their concentrations in the formulation, it is possible to obtain either a soft solid or a firm solid. In a preferred embodiment, the oil phase comprises a high molecular weight (MW) polyethylene. As used herein, the term “high molecular weight polyethylene” or “high MW polyethylene” means polyethylene having a molecular weight of 200 to 5000 daltons (Da). In a more preferred embodiment, the oil phase comprises high MW polyethylene having a molecular weight of about 500 Da. In another preferred embodiment, the oil phase comprises high MW polyethylene in an amount of about 5 to about 15 wt. % of the total first portion. In this regard, polyethylene can be used in smaller amounts as a structurant in the first portion than other structurants, such as stearyl alcohol. Stearyl alcohol is commonly used as a structurant in solid stick underarm products. However, stearyl alcohol has a tendency to leave visible white deposits on the skin, and the deposits can also transfer onto clothing when the clothing comes into contact with the skin. Accordingly, in another preferred embodiment, the oil phase comprises substantially no stearyl alcohol. The term “substantially no stearyl alcohol” as used herein means no stearyl alcohol or stearyl alcohol in an amount that is sufficiently small so that it would not cause visible white residue to deposit on skin and/or clothing after application of the antiperspirant product to the skin.

In accordance with another exemplary embodiment, when high MW polyethylene is used in the oil phase as a structurant, the oil phase also comprises at least one low MW synthetic wax. In addition to facilitating the high MW polyethylene by serving a structurant function, the low MW synthetic wax also improves the manufacturing processes of the antiperspirant products. Generally, polyethylene has a relatively high melting point (70-100° C.) and, thus, as described in more detail below, the oil phase of the emulsion portion must be heated to this high melting point to melt the polyethylene. However, this high temperature heating may result in higher manufacturing costs and unpredictable and/or non-repeatable yields of the antiperspirant product. The presence of an effective amount of low MW synthetic wax (synthetic wax having a molecular weight in the range of 1200-2900 Da) modifies the high MW polyethylene, lowering the melting point of the polyethylene. In an exemplary embodiment, the low MW synthetic wax is present in the oil phase in an amount of about 0 to about 3 wt. % of the total emulsion portion. In another exemplary embodiment, the low MW synthetic wax has a molecular weight of about 1800. In addition to improving hardness of the emulsion portion, the low MW synthetic wax reduces syneresis and tackiness and also has a high refractive index (R.I.) that minimizes and/or prevents a white residue on the skin by masking the antiperspirant metallic salt(s) that stays upon the skin upon evaporation of the carrier(s), described in more detail below. As used herein, the term “high refractive index” means an refractive index no less than about 1.4.

The oil phase further comprises at least one hydrophobic carrier. An example of suitable hydrophobic carriers includes liquid siloxanes and particularly volatile polyorganosiloxanes, that is, liquid materials having a measurable vapor pressure at ambient conditions. The polyorganosiloxanes can be linear or cyclic or mixtures thereof. The linear volatile silicones generally have viscosities of less than about 5 centistokes at 25° C., while the cyclic volatile silicones have viscosities under 10 centistokes. Preferred siloxanes include cyclomethicones, which have from about 3 to about 6 silicon atoms, such as cyclotetramethicone, cyclopentamethicone, and cyclohexamethicone, and mixtures thereof. The carrier also may comprise, additionally or alternatively, nonvolatile silicones such as dimethicone and dimethicone copolyols, which have from about 2 to about 9 silicon atoms. Examples of suitable dimethicone and dimethicone copolyols include polyalkyl siloxanes, polyalkylaryl siloxanes, and polyether siloxane copolymers.

The oil phase may also comprise a high R.I. hydrophobic compound. The high R.I. hydrophobic compound minimizes and/or prevents a white residue on the skin by masking the antiperspirant metallic salt that stays upon the skin upon evaporation of the carrier(s). Examples of high R.I. hydrophobic compounds for use in the antiperspirant products include C₁₂-C₁₅ alkyl benzoate, such as Finsolv TN® available from Innospec of the United Kingdom, PPG-14 butyl ether, and phenyl trimethicone. In a preferred embodiment, the oil phase comprises C₁₂-C₁₅ alkyl benzoate and, in a more preferred embodiment, the oil phase comprises C₁₂-C₁₅ alkyl benzoate in an amount of about 2 to about 12 wt. % of the total emulsion portion.

The emulsion portion of the antiperspirant product contemplated herein also may comprise additives, such as those used in conventional antiperspirants. For example, in addition to, or instead of, antiperspirant efficacy, the emulsion portion may comprise additives that cause the antiperspirant product to exhibit long-lasting fragrance, odor protection, bacteria control, and/or another desired purpose and/or function. These additives include, but are not limited to, fragrances, including encapsulated fragrances, dyes, pigments, preservatives, antioxidants, moisturizers, and the like. These optional ingredients can be included in the first portion in an amount of 0 to about 20 wt. %.

In an exemplary embodiment, one or both of the first and second portions 16 and 18 is/are a solid wax portion or portions of the antiperspirant product. The solid wax portion is a solid wax formula comprising an anhydrous, hydrophobic vehicle including a volatile silicone and/or high melting component. In one exemplary embodiment, the solid wax portion further comprises an active antiperspirant compound suspended in the anhydrous, hydrophobic vehicle. Any of the active antiperspirant compounds or salts listed above including for the emulsion portion may be used in the solid wax portion. In one exemplary embodiment, the same active antiperspirant compound or compounds is used in both the first portion 16 and the second portion 18. Alternatively, the second portion 18 may comprise a different active antiperspirant compound or compounds than the first portion 16. In one embodiment, the solid wax portion comprises the active antiperspirant compound in an amount of about 0 to about 25 wt. % (USP). In a preferred embodiment, the solid wax portion comprises the active antiperspirant compound in an amount of about 3 to about 25 wt. % (USP). More preferably, the solid wax portion comprises the active antiperspirant compound in an amount of about 8 to about 22 wt. % (USP).

The high melting components may include any material suitable for use in an antiperspirant stick that melts at a temperature of about 70° C. or higher. Typical of such materials are the high melting point waxes. These include beeswax, spermaceti, carnauba, bayberry, candelilla, montan, ozokerite, ceresin, paraffin waxes, semi-microcrystalline and microcrystalline waxes, hydrogenated jojoba oil, and hydrogenated castor oil (castor wax). The preferred wax is hydrogenated castor oil. Other suitable high melting components include various types of high melting gelling agents such as polyethylene-vinyl acetate copolymers, polyethylene homopolymers, 12-hydroxystearic acid, and substituted and unsubstituted dibenzylidene alditols. Typically, the high melting components comprise about 1 to about 25 wt. %, preferably about 2 to about 15 wt. %, of the composition. Volatile silicones include cyclomethicones and dimethicones, discussed above.

Other components may include, for example, non-volatile silicones, polyhydric alcohols having 3-6 carbon atoms and 2-6 hydroxy groups, fatty alcohols having from 12 to 24 carbon atoms, fatty alcohol esters, fatty acid esters, fatty amides, non-volatile paraffinic hydrocarbons, polyethylene glycols, polypropylene glycols, polyethylene and/or polypropylene glycol ethers of C₄-C₂₀ alcohols, polyethylene and/or polypropylene glycol esters of fatty acids, and mixtures thereof. The term “fatty” is intended to include hydrocarbon chains of about 8 to 30 carbon atoms, preferably about 12 to 18 carbon atoms.

Non-volatile silicones include polyalkylsiloxanes, polyalkylaryl siloxanes, and polyethersiloxanes with viscosities of about 5 to about 100,000 centistokes at 25° C., polymethylphenylsiloxanes with viscosities of about 15 to about 65 centistokes, and polyoxyall kylene ether dimethylsiloxane copolymers with viscosities of about 1200 to about 1500 centistokes.

Useful polyhydric alcohols include propylene glycol, butylenes glycol, dipropylene glycol and hexylene glycol. Fatty alcohols include stearyl alcohol, cetyl alcohol, myristyl alcohol, oleyl alcohol, and lauryl alcohol. Fatty alcohol esters include C₁₂₋₁₅ alcohols benzoate, myristyl lactate, cetyl acetate, and myristyl octanoate. Fatty acid esters include isopropyl palmitate, myristyl myristate, and glyceryl monostearate. Fatty amides include stearamide MEA, stearamide MEA-stearate, lauramide DEA, and myristamide MIPA.

Non-volatile paraffinic hydrocarbons include mineral oils and branched chain hydrocarbons with about 16 to 68, preferably about 20 to 40, carbon atoms. Suitable polyethylene glycols and polypropylene glycols will typically have molecular weights of about 500 to 6000, such as PEG-10, PEG-40, PEG-150 and PPG-20, often added as rheology modifiers to alter product appearance or sensory attributes.

Polyethylene and/or polypropylene glycol ethers or C₄-C₂₀ alcohols include PPG-10 butanediol, PPG-14 butyl ether, PPG-5-buteth-7, PPG-3-isostearth-9, PPG-3-myreth-3, oleth-10, and steareth-20. Polyethylene and/or polypropylene glycol esters of fatty acids include PEG-8 distearate, PEG-10 dioleate, and PPG-26 oleate. These are generally added to give emollient properties.

The above list of materials is by way of example only and is not intended to be a comprehensive list of all potential components of the antiperspirant products contemplated herein. Other high and low melting waxes, volatile and non-volatile compounds and other suitable components are readily identifiable to those skilled in the art. Of course, other ingredients such as colloidal silicas, particulate polyolefins, talcum materials, colorants and preservatives may also be included as desired.

In accordance with exemplary embodiments, a method for manufacturing the antiperspirant product illustrated in FIGS. 1 and 2 is shown in FIGS. 3-23. With reference to FIG. 23, the method generally denoted at 210 comprises mixing ingredients including a first ingredient to form the first portion (step 212). In one example, one or more premixes are prepared which are subsequently blended together or with other ingredients to form the first portion. For instance, an active antiperspirant premix may be prepared which is subsequently blended with other ingredients including a structurant, hot melt waxes, etc. to form a solid wax antiperspirant composition. In another example, the first ingredient, hydrophilic silica particles and water are mixed together, preferably using a low shear mixing process, until a homogeneous first ingredient premix is formed where the first ingredient is captured on a plurality of silica particles. Without being limited by theory, it is believed that the water facilitates the absorption and/or absorption of the first ingredient onto and/or into the hydrophilic silica particles during mixing. The first ingredient premix is then combined with one or more ingredients to form the first portion.

In an exemplary embodiment, various ingredients including a second ingredient are mixed together to form the second portion (step 214). In one example, one or more premixes are prepared which are subsequently blended together or with other ingredients to form the second portion. For instance, the second ingredient, hydrophilic silica particles and water may be mixed together, preferably using a low shear mixing process, until a homogeneous second ingredient premix is formed where the second ingredient is captured on a plurality of silica particles. The premix is then mixed with other ingredients including a structurant, hot melt waxes, etc. to form the second portion.

As discussed in further detail below with reference to FIGS. 3-22, the antiperspirant product is molded directly into a mold by depositing the first portion in molten form into the mold (step 216) and at least partially solidifying the first portion (step 218). The second portion is then deposited in molten form into the mold (step 220) and is at least partially solidify (222) to form the antiperspirant product. The mold may be used as the container for the antiperspirant product to form the antiperspirant composition 25 illustrated in FIG. 3. It will be appreciated, however, that the invention is not limited to use of the container as a mold and that any satisfactory mold may be used for manufacturing the antiperspirant product.

Referring to FIG. 3, the container 12 has an application end 24 and an opposite end 26. The container 12 also contains a factory seal 28, which is placed over the application surface 14 of antiperspirant product 10 to protect it during shipment and to render it tamper-proof prior to purchase, and a cover 30. The factory seal 28 is removed by the user, and the cover is used during storage of the product between uses. As the product is exhausted, it is advanced from the container by the user using advancement device 32, e.g., a screw mechanism as shown, at opposite end 26 of container 12.

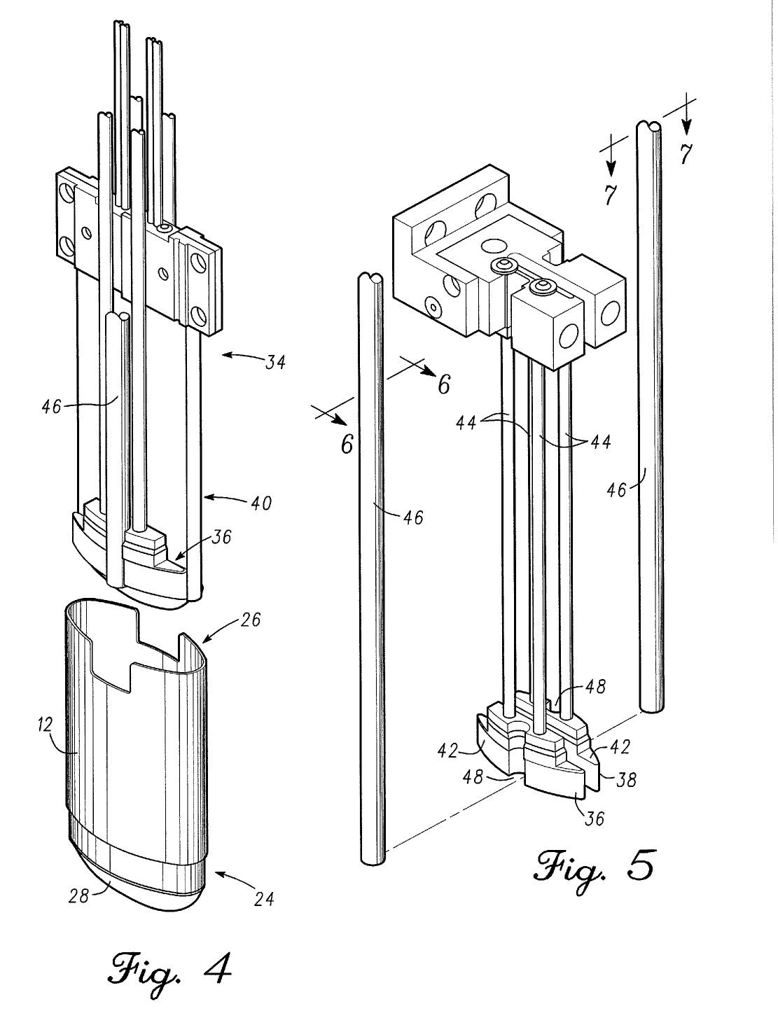

Referring to FIG. 4, a filling assembly 34 is positioned above opposite end 26 of an empty container 12. The factory seal 28 is in place, sealing the application end 24 of the container 12. The filling assembly 34 is lowered into the container 12 and is used to fill two compositions into the container, as will be described below with reference to FIGS. 13-18. The filling assembly 34 will first be described herein.

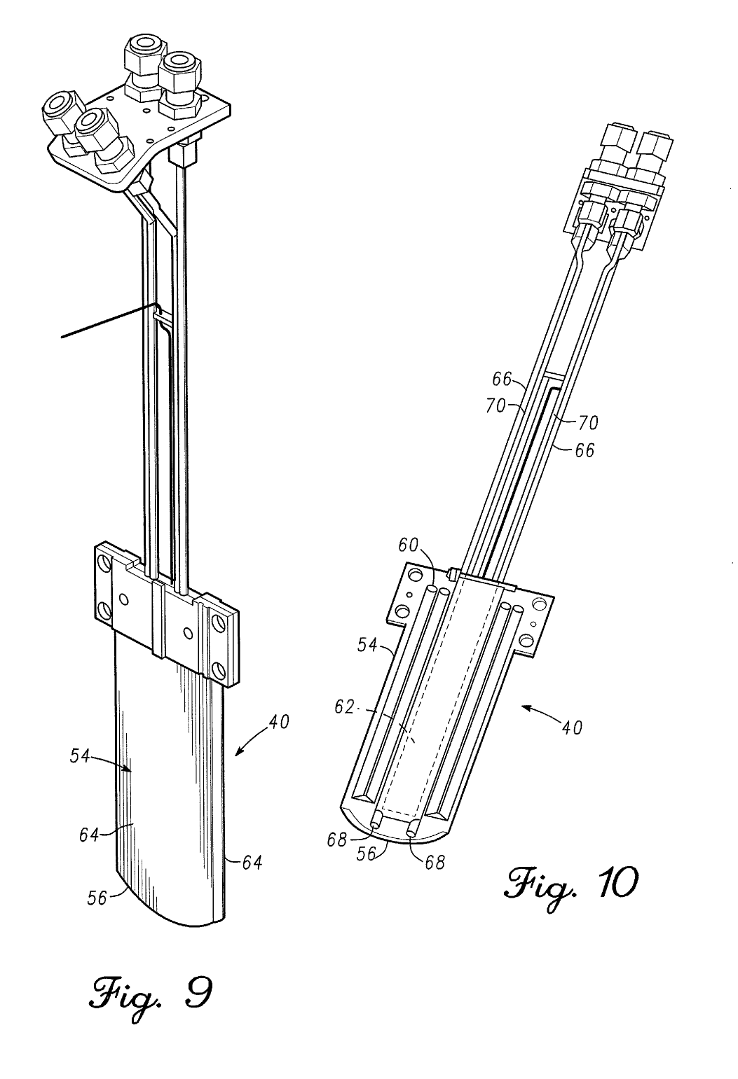

The components of filling assembly 34 are shown individually and in detail in FIGS. 5-10. The filling assembly consists of two outer nozzle assemblies 36 and 38, as shown in FIG. 5, and an inner nozzle assembly 40, as shown in FIG. 9. A first portion of the antiperspirant product is delivered by the outer nozzle assemblies 36 and 38, and a second portion is delivered by the inner nozzle assembly 40. The term “nozzle” as used herein refers to any device that is capable of delivering a fluid composition.

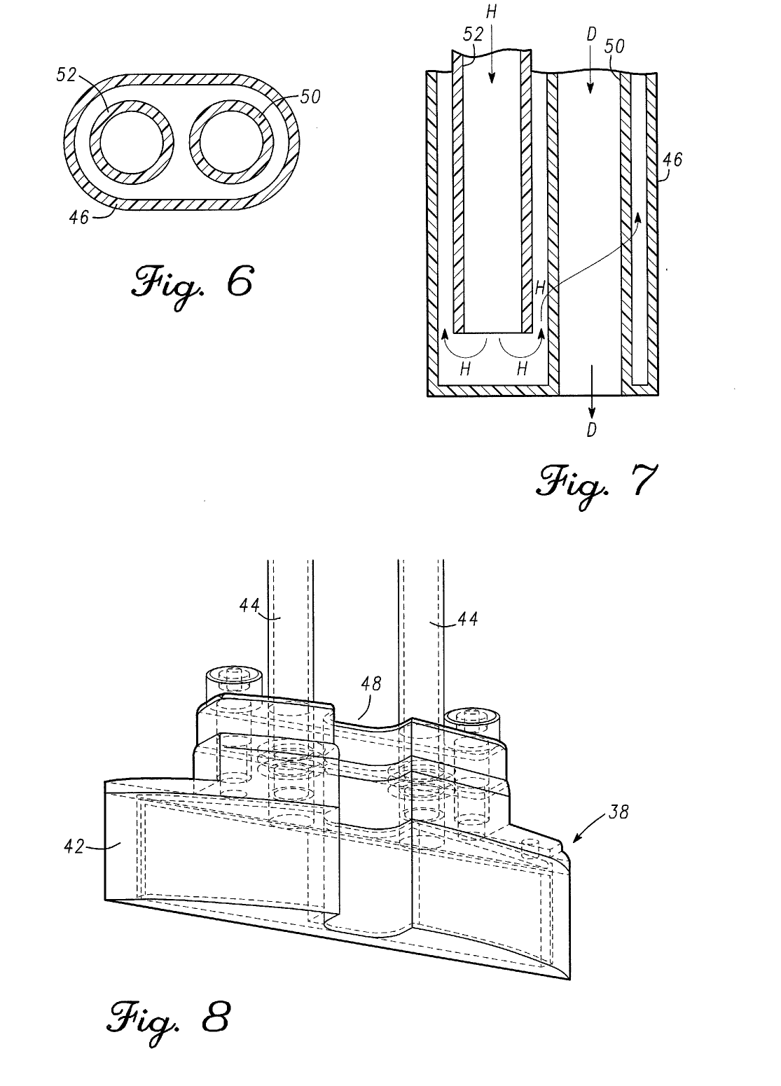

Each of the outer nozzle assemblies 36 and 38, one of which is shown in detail in FIG. 8, include a scraper body 42 that is mounted on two outer tubes 44. The scraper body is hollow, and is chilled by the circulation of cooling media. Its function will be discussed below. A center nozzle tube 46, disposed between the outer tubes 44, is retained in a groove 48 (FIG. 8) in the scraper body (center nozzle tube 46 is omitted in FIG. 8 for clarity). The two outer tubes 44 support the scraper body 42, allowing it to be moved vertically, and circulate cooling media to and from the scraper body. As shown in FIGS. 6 and 7, the center nozzle tube 46 consists of a delivery tube 50 and a heating tube 52. The heating tube circulates heating media (arrows H, FIG. 7) to maintain the first portion in a molten state as it is delivered thought the delivery tube 50 (arrows D, FIG. 7). A temperature sensor, e.g., a thermocouple, thermistor, or the like (not shown), may be provided on one or both of the scraper bodies to measure the temperature of the scraper body.

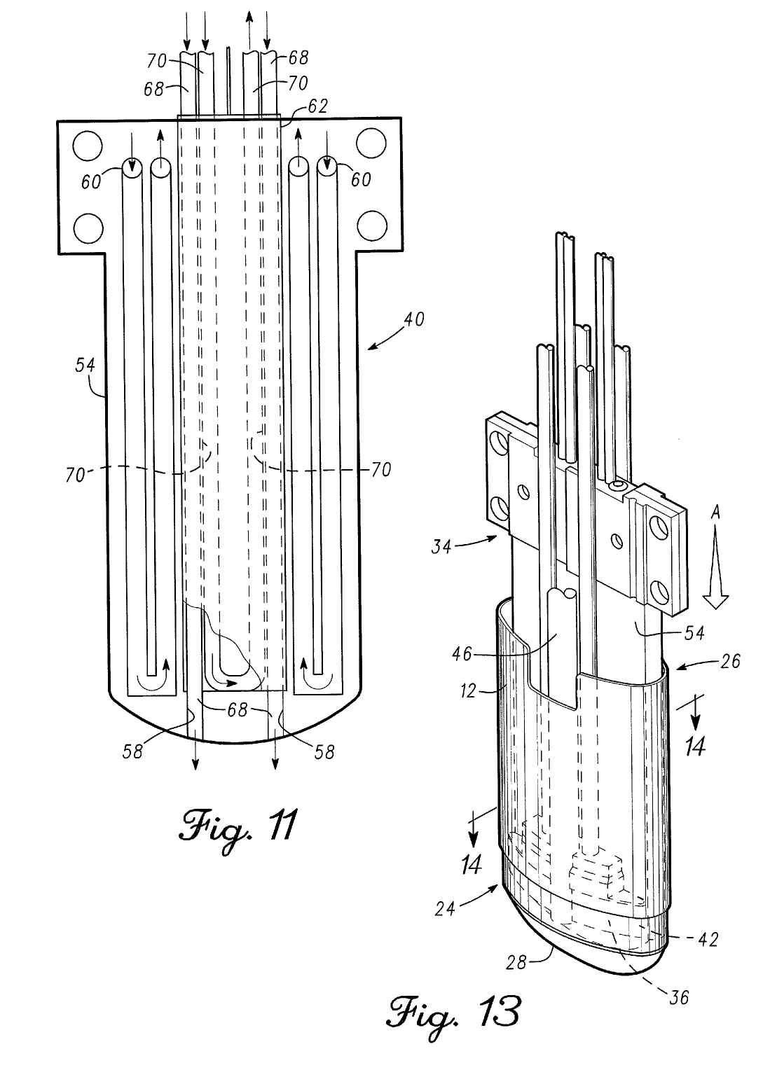

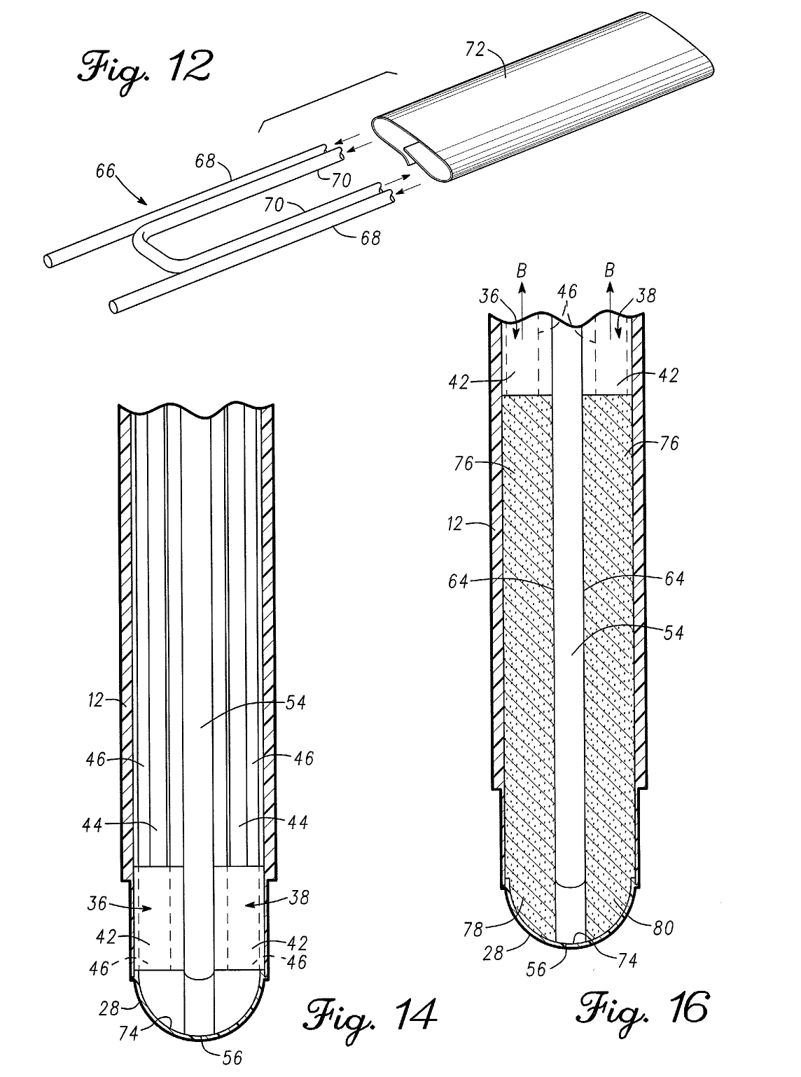

The inner nozzle assembly 40 includes a housing 54 that provides a molding surface for the first portion, as will be discussed below. The housing 54 includes a curved leading edge 56 shaped to sealingly engage the inner surface of the factory seal 28. If desired, the leading edge 56 may be a relatively sharp edge to provide a concentrated pressure against the factory seal 28. The interior of the housing 54 is shown in FIGS. 10, 11, and 12. The housing 54 defines a pair of delivery channels 58, a pair of substantially U-shaped cooling channels 60, and a central rectangular channel 62. The cooling channels circulate cooling media to chill the outer surface 64 of housing 54. The central rectangular channel 62 receives an assembly 66, shown in FIG. 12, which includes a pair of delivery tubes 68 brazed to a substantially U-shaped heating tube 70. The heating tube 70 circulates heating media to heat the second phase as it is being delivered through the delivery tubes 68. The assembly 66 is wrapped in insulation 72 (FIG. 12) to insulate it within the chilled housing. The inner nozzle assembly 40 may also include one or more temperature sensors (not shown) to determine the heating and/or cooling temperatures.

The process of filling the container 12, and thus molding the antiperspirant product 10, is shown in FIGS. 13-18. First, as shown in FIGS. 13 and 14, the filling assembly 34 is inserted into the container 12, through the opposite end 26 (arrow A), until the leading edge 56 of the housing 54 contacts the inner surface 74 of the factory seal 28. At this stage of the process, both the inner nozzle assembly 40 and the outer nozzle assemblies 36 and 38 are fully extended into the container 12. Although the cover 30 is omitted in FIGS. 4 and 13-18, for clarity, the cover is in place during the molding process. Cover 30 provides a flat surface on which the container can rest during filling, and also holds the factory seal in place against the downward pressure exerted by the inner nozzle assembly.

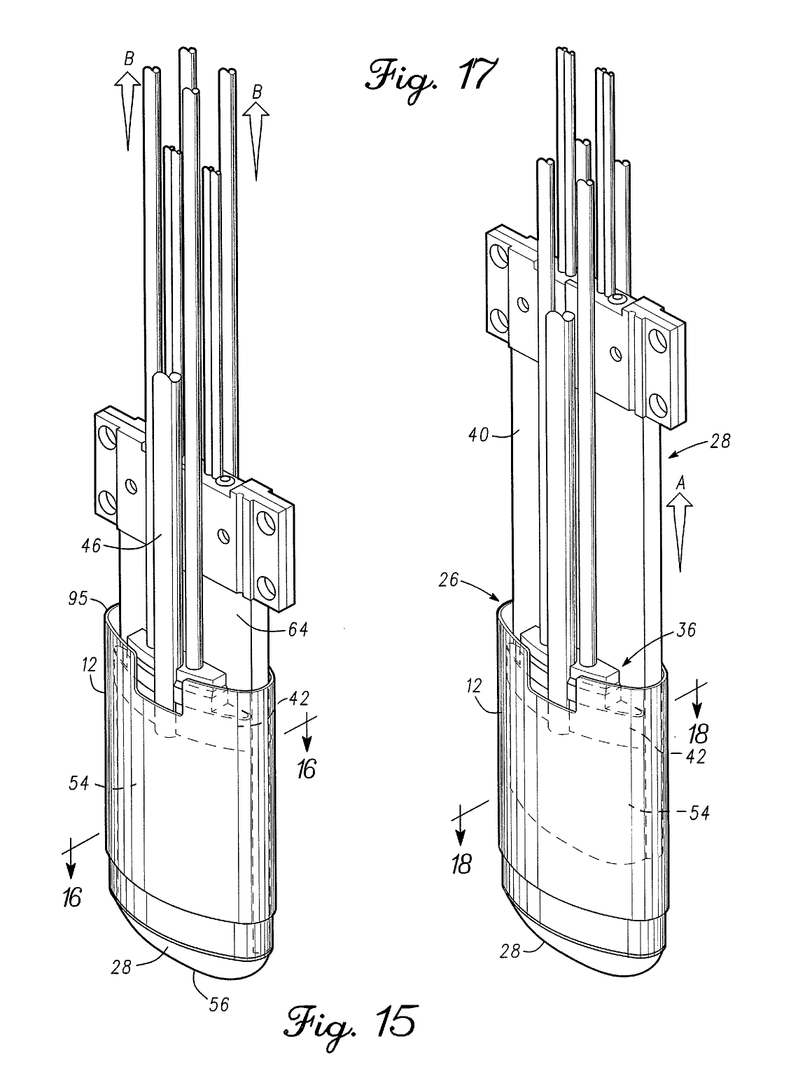

Next, as shown in FIGS. 15 and 16, the first portion, in liquid form (herein “the first fluid portion”), of the antiperspirant product is delivered to the container 12 to the open spaces on both sides of housing 54. Accordingly, to deliver the first fluid portion to the container in liquid form, the first fluid portion can be maintained at a temperature in the range of about 75 to about 80° C., for example, about 75° C. The first fluid portion 76 is delivered from delivery tubes 50 of outer nozzle assemblies 36 and 38, while the outer nozzle assemblies 36 and 38 are being simultaneously raised, as indicated by the arrows B. During delivery of the first fluid portion, the inner nozzle assembly 40 is maintained in its lowered position so that leading edge 56 provides a seal against the inner surface 74 of factory seal 28 to prevent first fluid portion 76 from flowing under the leading edge 56 and so that the outer surface 64 of the housing 54 provides a molding surface. Sealing is provided by the curved surface of the leading edge 56, which corresponds closely to the curvature of the surface 74 of the factory seal 28. Sealing can be enhanced by applying downward pressure to the inner nozzle assembly 40 during delivery of the first portion 16. The first fluid portion is molten, so that it is sufficiently fluid for delivery, but will solidify quickly as it cools. Because the outer surface 64 is chilled, the first fluid portion 76 will solidify relatively quickly.

The container 12, filled with the first fluid portion 76, is shown in FIG. 16. After the container 12 has been filled to a desired level, the first fluid portion 76 is allowed to solidify sufficiently so that a skin or thin solid layer will form to prevent the first fluid portion 76 from mixing with a second fluid portion. A skin thickness of from about 1 to about 2 millimeters (mm) is generally sufficient, typically requiring a dwell time of from about 1 to about 10 seconds, preferably from about 2 to about 6 seconds at about −10 to +20° C. The skin will form adjacent the surface 64 of the housing 54 due to the chilling of the surface 64. The dwell time will depend on the temperature to which the surface 64 of the housing 54 is cooled, and the temperature of the molten material when it is delivered. The resulting two regions 78 and 80 of the first fluid portion 76 (FIG. 12) will define the regions having the surfaces 20 of first portion 16 (FIG. 1).

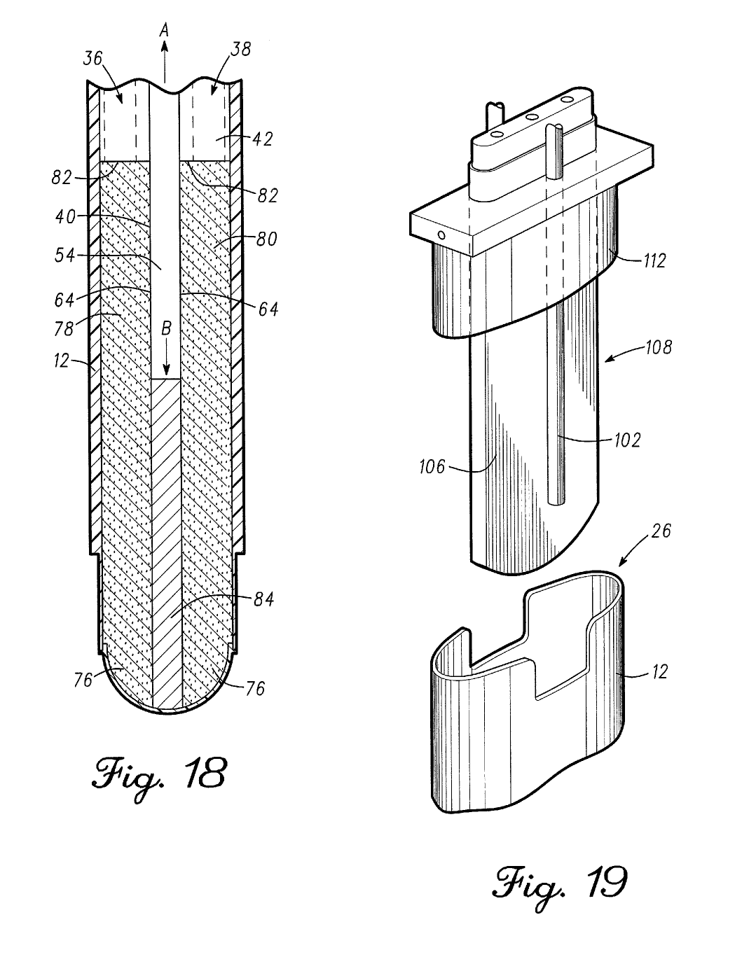

During this dwell time, and then during the subsequent filling step described below, the outer nozzle assemblies 36 and 38 are maintained in a position, shown in FIG. 18, at which a lower surface 81 of each scraper body 42 is in contact with the top surface of the regions 78 and 80 of the first fluid portion 76. In this position, the chilled scraper bodies serve several functions: (a) they aid in solidification of the top surface of the first fluid portion 76, (b) they hold the first fluid portion 76 in the container during the next step, described below, and (c) they scrape the outer surface 64 of housing 54 during the next step, which helps the solidified skin to release from surface 64. With respect to the first function, the chilling of the top surface of the first fluid portion 76 causes a skin to form at the top surface, which extends from the inner wall of the container and thus provides lateral support to the regions 78 and 80, preventing them from collapsing or leaning inward. If additional lateral support is desired, the upward movement of the outer nozzle assemblies during the filling process can be interrupted, e.g., for about one second, at an intermediate position within the container. The intermediate position may be about halfway up. This brief pause in the filling operation will allow a skin to form under the scraper bodies 42 at this point, providing additional lateral support to the regions 78 and 80.

Referring to FIGS. 17 and 18, when the first fluid portion 76 has sufficiently solidified (formed a skin), the inner nozzle assembly 40 is moved upwards (arrow A), out of the container. As the inner nozzle assembly 40 moves upward, a second fluid portion 84 is delivered to the space that becomes available between regions 78 and 80 of the first fluid portion 76 as the housing 54 is removed, as indicated by the arrow B in FIG. 18. The second fluid portion 84 is delivered through delivery channels 58 of the inner nozzle assembly 40. The second fluid portion will define the second portion 18 of the finished product (FIG. 1).

The retraction of the inner nozzle assembly 40 is coordinated with the delivery of the second fluid portion 84 so that the volume vacated by the nozzle is immediately filled with the liquid volume that is being delivered. This prevents damage to the weak skin that supports the regions 78 and 80 and prevents intermingling of the fluid portions. This coordination may be achieved, e.g., by electronically linking servo motors that control a delivery pump to a screw that retracts the nozzle assembly.

During delivery of second fluid portion 84, the chilled scraper bodies prevent the regions 78 and 80 from being lifted upwards by friction exerted by the outer surface 64 of housing 54, helping the skin to release from the surface 64, and scraping off any of the first fluid portion 76 that adheres to outer surface 64 during removal of housing 54. This leaves the outer surface 64 of housing 54 clean prior to filling of a new container.

The steps shown in FIGS. 17 and 18 complete the molding process and the manufacturing of the antiperspirant composition 25 illustrated in FIG. 3. Solidification of the first and second fluid portions is completed by cooling the product, for example, by passing the filled container through a forced air tunnel operating at between about 10 to about 25° C. The finished product (FIG. 3) is completed by sealing the open opposite end 26 with a package base (not shown) that includes advancement device 32.

Suitable materials for housing 54 include metals such as stainless steel, aluminum alloys, copper or beryllium. Coated metals can also be used, e.g., stainless steel coated with titanium nitride, chromium, or electroless nickel with a polytetrafluoroethylene (PTFE) infusion; aluminum coated with aluminum oxide hardcoat anodizing, hardcoat anodizing with a PTFE infusion, or electroless nickel with or without a PTFE infusion; or aluminum plated with nickel or chrome. The housing may be coated with a release coating such as liquid silicone to enhance release of the skin.

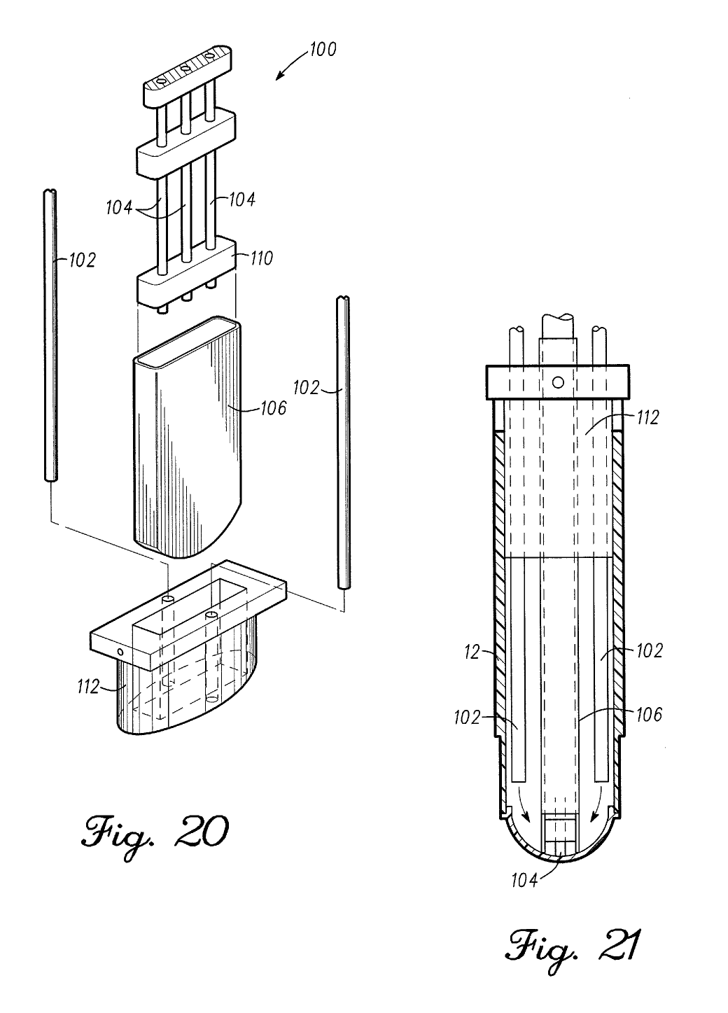

An alternative molding process can be performed using the filling assembly 108 shown in FIGS. 19 and 20. In this embodiment, the inner nozzle assembly 100 includes a plurality of delivery tubes 104 (FIG. 20) surrounded by a housing 106 that can be raised and lowered independently of the delivery tubes 104. The outer nozzle assemblies discussed above are replaced by the outer nozzles 102 and the scraper block 112, with the scraper block 112 performing the functions described above with reference to the scraper bodies 42. (If desired, the outer nozzle assemblies discussed above may be used in this embodiment.)

Because, in this embodiment, the housing 106 can be moved independently of the delivery tubes 104, the first and second fluid portions can be filled in any desired order, or simultaneously. If they are filled simultaneously, as shown in FIG. 21, the housing 106 would be left in place for a sufficient length of time to allow at least one of the compositions to form a skin to prevent intermingling of the two fluid portions. Thus, the housing 106 may be moved upward slowly, a few seconds behind the nozzles.

In this embodiment, it is generally useful that the inside surface of the housing 106 be scraped. To accomplish this, the member 110 (FIG. 20) is mounted on the delivery tubes 104, and shaped to closely fit the interior of the housing 106 so that, when the delivery tubes 104 are moved vertically relative to the housing 106, the member 110 will scrape the inner surface of the housing.

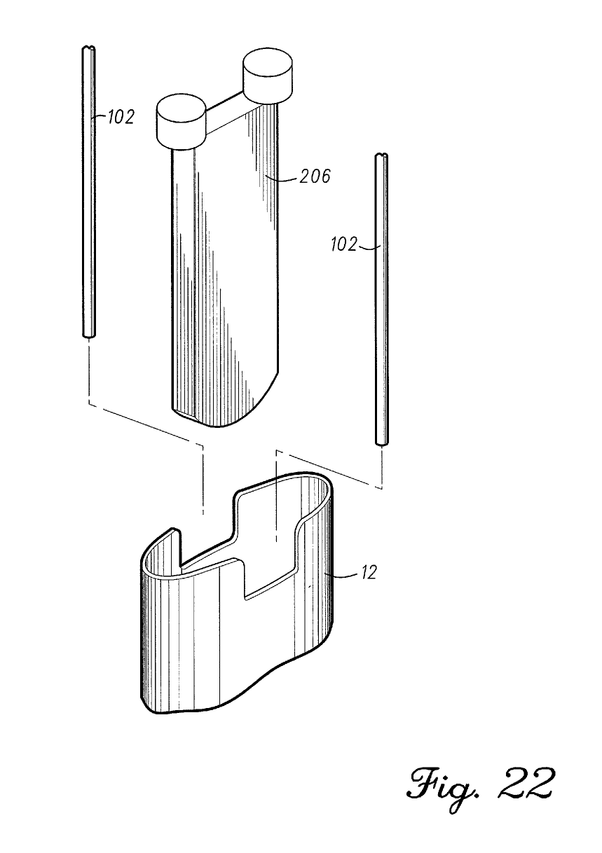

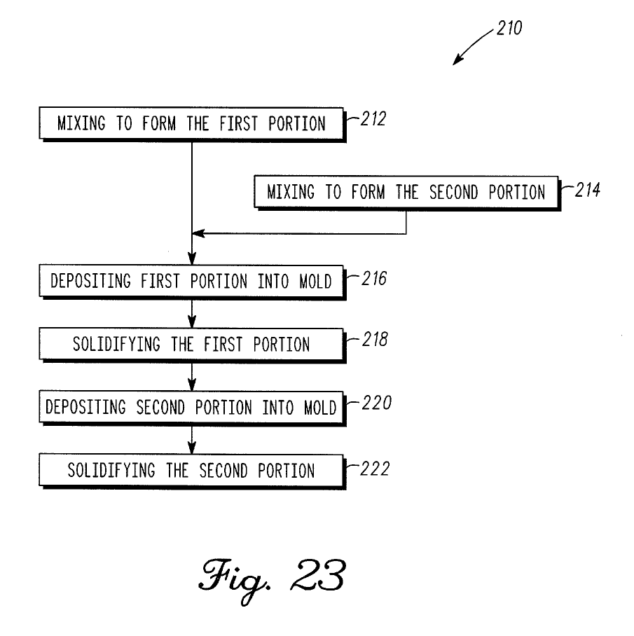

Another alternative molding process can be performed as shown in FIG. 22. In this embodiment, a solid housing 206 is used and is seated directly into the container 12. The outer nozzles 102 fill the first fluid portion as shown in FIG. 21. The solid housing 206 remains in place for a sufficient length of time to at least allow the first fluid portion to partially solidify and form a skin. Preferably, solidification of the first fluid portion may be facilitated by passing the container-mold assembly through a forced air tunnel operating at between about 10 to about 25° C. The solid housing 206 may be moved upward slowly, a few seconds behind the nozzles 102, and then the solid housing 206 may be cleaned after removal from the container 12.

The delivery tubes 104, illustrated in FIG. 20, are then positioned between the solidified outer first portions and the second fluid portion is delivered into the container 12. Solidification of the second fluid portion may be facilitated by again passing the container-mold assembly through the forced air tunnel.

The following are examples of the first and second portions of the antiperspirant product in accordance with the present invention with each of the components set forth in weight percent. The examples are provided for illustration purposes only and are not meant to limit the various embodiments of the antiperspirant product in any way. All materials are set forth in weight percent.

### Example 1

### Active Antiperspirant Salt/Release Enhancer

Outer Portion—Solid Wax Formulation with Active Antiperspirant Salt:

| Active Premix - Ingredient | Wt. % |
|---|---|
| Aluminum Zirconium Trichlorohydrex GLY | 30-70 |
| Cyclopentasiloxane | 30-70 |
| Aerosil 300 (Silica) | 0.1-4 |
| Silica Dimethyl Silylate | 0.1-4 |
| Total | 100.0 |

| Outer Portion - Ingredient | Wt. % |
|---|---|
| Active Premix | 30-70 |
| DC 245 Fluid | 2-15 |
| Castor Oil, Hydrogenated | 2-8 |
| Myristal Myristate | 1-4 |
| Fluid AP, Low Odor Bulk | 8-15 |
| Stearyl Alcohol, Solid | 15-22 |
| Total | 100.0 |

Inner Portion—Solid Wax Formulation with Release Enhancer:

| Release Enhancement Premix- Ingredient | Wt. % |
|---|---|
| Organofunctional Siloxane | 10-50 |
| Linoleamidopropyl PG Dimonium | 10-50 |
| Chloride Phosphate Dimethicone | |
| Water | 5-20 |
| Sipernat ® 22S | 25-50 |
| Total | 100.0 |

| Inner Portion - Ingredient | Wt. % |
|---|---|
| DC 245 Fluid | 30-70 |
| Stearyl Alcohol, Solid | 15-22 |
| Castor Oil, Hydrogenated | 2-8 |
| Myristyl Myristate | 1-4 |
| Fluid AP, Low Odor Bulk | 8-15 |
| Fragrance | 2-8 |
| Encapsulated Fragrance | 1-4 |
| Protectate | 0.1-2 |
| Release Enhancement Premix | 5-25 |
| Total | 100.0 |

### Example 2

### Active Antiperspirant Salt/Release Enhancer

Outer Portion—Emulsion Formulation with Active Antiperspirant Salt and Release Enhancer:

| | Wt. % |
|---|---|
| Water Phase - Ingredient | |
| Aluminum Zirconium Trichlorohydrex GLY | 10-50 |
| Water | 10-50 |
| Propylene Glycol | 5-15 |
| Oil Phase - Ingredient | |
| Cyclomethicone | 10-25 |
| C₁₂₋₁₅ Alkyl Benzoate | 5-15 |
| Cetyl PEG/PPG-10/1 Dimethicone | 1-4 |
| Polyethylene Wax | 8-15 |
| PEG-25 Propylene Glycol Stearate | 2-8 |
| Synthetic Wax | 0.01-2 |
| Total (Water Phase + Oil Phase) | 100.0 |

Inner Portion—Solid Wax Formulation with Active Antiperspirant Salt:

| Premix for Wax Phase - Ingredient | Wt. % |
|---|---|
| Aluminum Zirconium Trichlorohydrex GLY | 30-70 |
| Cyclopentasiloxane | 30-70 |
| Aerosil 300 (Silica) | 0.1-4 |
| Silica Dimethyl Silylate | 0.1-4 |
| Total | 100.0 |

| Inner Portion (Wax Phase) - Ingredient | Wt. % |
|---|---|
| Premix for Wax Phase | 30-70 |
| Cyclopentasiloxane | 8-25 |
| Stearyl Alcohol | 15-22 |
| Castor Oil, Hydrogenated | 2-8 |
| Myristyl Myristate | 1-4 |
| PPG-14 Butyl Ether | 8-15 |
| Chromium Green Hydroxide | 0.01-1 |
| Fragrance | 2-8 |
| Encapsulated Fragrance | 1-4 |
| Protectate | 0.1-2 |
| Total | 100.0 |

### Example 3

### Active Antiperspirant Salt/Activity Enhancement Agent

Outer Portion—Emulsion Formulation with Active Antiperspirant Salt and Activity

Enhancement Agent:

| | Wt. % |
|---|---|
| Water Phase - Ingredient | |
| Aluminum Zirconium Trichlorohydrex GLY | 10-40 |
| Water | 10-40 |
| Propylene Glycol | 5-15 |
| Activity Enhancement Premix- Ingredient | |
| Calcium Chloride | 2-15 |
| Glycine | 2-15 |
| Water | 30-70 |
| Sipernat ® 22S | 25-50 |
| Total (Water Phase + Oil Phase) | 100.0 |

| Oil Phase - Ingredient | Wt. % |
|---|---|
| Cyclohexasiloxane | 5-25 |
| C₁₂₋₁₅ Alkyl Benzoate | 5-15 |
| Cetyl PEG/PPG-10/1 Dimethicone | 1-4 |
| Polyethylene Wax | 8-15 |
| Activity Enhancement Premix | 2-8 |
| Synthetic Wax | 0.01-2 |
| Total | 100.0 |

Inner Portion—Solid Wax Formulation with Active Antiperspirant Salt:

| Premix for Wax Phase - Ingredient | Wt. % |
|---|---|
| Aluminum Zirconium Trichlorohydrex GLY | 30-70 |
| Cyclopentasiloxane | 30-70 |
| Aerosil 300 (Silica) | 0.1-4 |
| Silica Dimethyl Silylate | 0.1-4 |
| Total | 100.0 |

| Inner Portion (Wax Phase) - Ingredient | Wt. % |
|---|---|
| Premix for Wax Phase | 30-70 |
| Cyclopentasiloxane | 8-25 |
| Stearyl Alcohol | 15-22 |
| Castor Oil, Hydrogenated | 2-8 |
| Myristyl Myristate | 1-4 |
| PPG-14 Butyl Ether | 8-15 |
| Chromium Green Hydroxide | 0.01-1 |
| Fragrance | 2-8 |
| Encapsulated Fragrance | 1-4 |
| Protectate | 0.1-2 |
| Total | 100.0 |

### Example 4

### Antibacterial Agent/Solubilizer

Outer Portion—Solid Wax Formulation with Microencapsulated Hydrophobic Antibacterial Agent:

| Active Premix - Ingredient | Wt. % |
|---|---|
| Aluminum Zirconium Trichlorohydrex GLY | 30-70 |
| Cyclopentasiloxane | 30-70 |
| Aerosil 300 (Silica) | 0.1-4 |
| Silica Dimethyl Silylate | 0.1-4 |
| Total | 100.0 |

| Outer Portion - Ingredient | Wt. % |
|---|---|
| Active Premix | 30-70 |
| DC 245 Fluid | 2-15 |
| Castor Oil, Hydrogenated | 2-8 |
| Myristal Myristate | 1-4 |
| Fluid AP, Low Odor Bulk | 8-15 |
| Microencapsulated (Benzalkonium | 1-8 |
| Chloride/Ethanol) | |
| Stearyl Alcohol, Solid | 15-22 |
| Total | 100.0 |

Inner Portion—Solid Wax Formulation with Microencapsulated Solubilizer:

| Inner Portion - Ingredient | Wt. % |
|---|---|
| Active Premix | 30-70 |
| Cyclopentasilioxane | 5-15 |
| Stearyl Alcohol, Solid | 15-22 |
| Castor Oil, Hydrogenated | 2-8 |
| Myristyl Myristate | 1-4 |
| PPG-14 Butyl Ether | 8-15 |
| Fragrance | 2-8 |
| SYM 388782 Triton 70% Incap 197341/PSM/60 | 1-4 |
| Protectate | 0.1-2 |
| Microencapsulated (PPG-17/PEG-8) | 5-25 |
| Total | 100.0 |

### Example 5

### Fragrance Extract/Solvent

Outer Portion—Solid Wax Formulation with Fragrance Extract:

| Active Premix - Ingredient | Wt. % |
|---|---|
| Aluminum Zirconium Trichlorohydrex GLY | 30-70 |
| Cyclopentasiloxane | 30-70 |
| Aerosil 300 (Silica) | 0.1-4 |
| Silica Dimethyl Silylate | 0.1-4 |
| Total | 100.0 |

| Outer Portion - Ingredient | Wt. % |
|---|---|
| Active Premix | 30-70 |
| DC 245 Fluid | 2-15 |
| Castor Oil, Hydrogenated | 2-8 |
| Myristal Myristate | 1-4 |
| Fluid AP, Low Odor Bulk | 8-15 |
| Stearyl Alcohol, Solid | 15-22 |
| Fragrance Resin/Wax | 1-8 |
| Total | 100.0 |

Inner Portion—Solid Wax Formulation with Silica Suspension of Solvent (Ethanol):

| Solvent Premix- Ingredient | Wt. % |
|---|---|
| Ethanol | 30-70 |
| Water | 5-25 |
| Sipernat ® 22S | 20-50 |
| Total | 100.0 |

| Inner Portion - Ingredient | Wt. % |
|---|---|
| Active Premix | 30-70 |
| DC 245 Fluid | 1-4 |
| Stearyl Alcohol, Solid | 15-22 |
| Castor Oil, Hydrogenated | 2-8 |
| Myristyl Myristate | 1-4 |
| Fluid AP, Low Odor Bulk | 8-15 |
| Fragrance | 0.1-5 |
| Encapsulated Fragrance | 1-4 |
| Protectate | 0.1-2 |
| Solvent Premix | 10-20 |
| Total | 100.0 |

### Example 6

### Dye Transfer Inhibitor/Color Additive

Outer Portion—Solid Wax Formulation with Color Additives Including Pigment with Free Dye:

| Active Premix - Ingredient | Wt. % |
|---|---|
| Aluminum Zirconium Trichlorohydrex GLY | 30-70 |
| Cyclopentasiloxane | 30-70 |
| Aerosil 300 (Silica) | 0.1-4 |
| Silica Dimethyl Silylate | 0.1-4 |
| Total | 100.0 |

| Outer Portion - Ingredient | Wt. % |
|---|---|
| Active Premix | 30-70 |
| DC 245 Fluid | 2-15 |
| Castor Oil, Hydrogenated | 2-8 |
| Myristal Myristate | 1-4 |
| Fluid AP, Low Odor Bulk | 8-15 |
| Stearyl Alcohol, Solid | 15-22 |
| FD&C Blue Lake #1 | 0.01-2 |
| Total | 100.0 |

Inner Portion—Solid Wax Formulation with Silica Suspension of Dye Transfer Inhibitor:

| Dye Transfer Inhibitor Premix- Ingredient | Wt. % |
|---|---|
| Poly(N-vinylpyrrolidone) | 20-50 |
| Water | 5-50 |
| Sipernat ® 22S | 20-50 |
| Total | 100.0 |

| Inner Portion - Ingredient | Wt. % |
|---|---|
| Active Premix | 30-70 |
| DC 245 Fluid | 2-15 |
| Stearyl Alcohol, Solid | 15-22 |
| Castor Oil, Hydrogenated | 2-8 |
| Myristyl Myristate | 1-4 |
| Fluid AP, Low Odor Bulk | 8-15 |
| Fragrance | 2-8 |
| Encapsulated Fragrance | 1-4 |
| Protectate | 0.1-2 |
| Dye Transfer Inhibitor Premix | 5-15 |
| Total | 100.0 |

### Example 7

### Dye Transfer Inhibitor/Color Additive

Outer Portion—Emulsion Formulation with Water Soluble Dye Transfer Inhibitor:

| | Wt. % |
|---|---|
| Water Phase - Ingredient | |
| Aluminum Zirconium Trichlorohydrex GLY | 10-40 |
| Water | 10-40 |
| Poly(N-vinylpyrrolidone) | 1-10 |
| Propylene Glycol | 5-15 |
| Oil Phase - Ingredient | |
| Cyclomethicone | 10-40 |
| C₁₂₋₁₅ Alkyl Benzoate | 5-15 |
| Cetyl PEG/PPG-10/1 Dimethicone | 1-4 |
| Polyethylene Wax | 8-15 |
| Synthetic Wax | 0.01-2 |
| Total (Water Phase + Oil Phase) | 100.0 |

Inner Portion—Solid Wax Formulation with Color Additives Including Pigment with Free Dye:

| Premix for Wax Phase - Ingredient | Wt. % |
|---|---|
| Aluminum Zirconium Trichlorohydrex GLY | 30-70 |
| Cyclopentasiloxane | 30-70 |
| Aerosil 300 (Silica) | 0.1-4 |
| Silica Dimethyl Silylate | 0.1-4 |
| Total | 100.0 |

| Inner Portion (Wax Phase) - Ingredient | Wt. % |
|---|---|
| Premix for Wax Phase | 30-70 |
| Cyclopentasiloxane | 2-20 |
| Stearyl Alcohol | 15-22 |
| Castor Oil, Hydrogenated | 2-8 |
| Myristyl Myristate | 1-4 |
| FD&C Blue Lake #1 | 0.1-2 |
| Fragrance | 2-8 |
| Encapsulated Fragrance | 1-4 |
| Protectate | 0.1-2 |
| Total | 100.0 |

Accordingly, antiperspirant products that exhibit antiperspirant efficacy and that also provide other predetermined effects to a user upon application and or upon perspiration have been described. The various embodiments of the antiperspirant products comprise a first portion containing a first ingredient and a second portion containing a second ingredient that is effective to interact with the first ingredient to produce the predetermined effect. The first and second portions are macroscopically separated from each other. By keeping the first and second ingredients separated in each of their corresponding portions, the predetermined effects that are produced from the interaction of the first and second ingredients are better preserved for end use. The first portion, the second portion, or both portions of the antiperspirant product can provide antiperspirant efficacy. Upon application, the two portions combine to provide a user with an antiperspirant product that exhibits one or more predetermined effects at the time of application, throughout the day, and/or when it is needed most, e.g. when the user perspires. Various non-limiting examples of the predetermined effects produced by interaction of the first and second ingredients include enhanced wetness protection, antibacterial protection, enhanced fragrance, introduced staining by color additives in the antiperspirant product so that the color additives do not transfer to the user's cloths or otherwise.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A personal care product comprising:
a container; and
an antiperspirant product housed within the container, the antiperspirant product comprising:
a first portion comprising a first ingredient; and
a second portion macroscopically separated from the first portion, the second portion having a composition different from the first portion and comprising a second ingredient effective to interact with the first ingredient to produce a predetermined effect.

2. The personal care product according to claim 1, wherein the first ingredient is a material selected from the group consisting of an active antiperspirant salt, an antibacterial agent, a fragrance extract, and a dye transfer inhibitor, and the second ingredient is a material selected from the group consisting of a release enhancer, a color additive, and an activity enhancement agent, and wherein the first ingredient, the second ingredient, or a combination thereof is adsorbed onto surfaces of silica, absorbed into pores of the silica, or a combination thereof.

3. The personal care product according to claim 1, wherein the first ingredient, the second ingredient, or a combination thereof is encapsulated into rupturable membranes.

4. The personal care product according to claim 1, wherein the first ingredient is an active antiperspirant salt, the second ingredient is a release enhancer and the predetermined effect is enhanced wetness protection.

5. The personal care product according to claim 4, wherein the active antiperspirant salt comprises a material selected from the group consisting of aluminum zirconium pentachlorohydrex GLY, aluminum zirconium trichlorohydrex GLY, aluminum chlorohydrate, and mixtures thereof, and the release enhancer comprises a material selected from the group consisting of alkoxylated compounds, ethoxylated compounds, PEG-25 propylene glycol stearate, ceteareth-12, ceteareth-20, ceteareth-30, PPG-3 benzyl ether ethylhexanoate, organofunctional siloxane, glyceral stearate, PEG-100 stearate, C20-40 pareth-10, phospholipids, linoleamidopropyl PG-dimonium chloride phosphate dimethicone, hydrogenated lecithin, unhydrogenated lecithin, phosphatidylcholine, phosphatidylethanolamine, phosphatidylerine, phosphatidylglycerol, cardiolipin, and mixtures thereof.

6. The personal care product according to claim 4, wherein the first portion comprises the active antiperspirant salt in an amount of from about 10 to about 50 weight percent (USP), and the second portion comprises the release enhancer in an amount of from about 0.1 to about 24 weight percent.

7. The personal care product according to claim 1, wherein the first ingredient is an antibacterial agent, the second ingredient is a solubilizer and the predetermined effect is antibacterial protection.

8. The personal care product according to claim 7, wherein the antibacterial agent comprises a material selected from the group consisting of triclocarban, triclosan, benzalkonium chloride, phenoxy ethanol, chlorohexidine, and mixtures thereof, and the solubilizer comprises a material selected from the group consisting of polyethylene glycols, polypropylene glycols, polyglycols, and mixtures thereof.

9. The personal care product according to claim 7, wherein the first portion comprises the antibacterial agent in an amount of from about 0.1 to about 6 weight percent, and the second portion comprises the solubilizer in an amount of from about 0.3 to about 14 weight percent.

10. The personal care product according to claim 1, wherein the first ingredient is a fragrance extract, the second ingredient is a solvent and the predetermined effect is an enhanced fragrance.

11. The personal care product according to claim 10, wherein the fragrance extract comprises a material selected from the group consisting of absolutes, crystalline, resinoids, resins, waxes, and mixtures thereof, and the solvent is ethanol.

12. The personal care product according to claim 10, wherein the first portion comprises the fragrance extract in an amount of from about 0.1 to about 6 weight percent, and the second portion comprises the solvent in an amount of from about 0.3 to about 12 weight percent.

13. The personal care product according to claim 1, wherein the first ingredient is a dye transfer inhibitor, the second ingredient is a color additive and the predetermined effect is reduced staining by the color additive.

14. The personal care product according to claim 13, wherein the dye transfer inhibitor comprises a material selected from the group consisting of poly(N-vinylpyrrolidone), poly(vinylpyridine N-oxide), and mixtures thereof, and the color additive comprises a material selected from the group consisting of dyes, pigments, and mixtures thereof.

15. The personal care product according to claim 13, wherein the first portion comprises the dye transfer inhibitor in an amount of from about 0.1 to about 10 weight percent, and the second portion comprises the color additive in an amount of from about 0.01 to about 3 weight percent.

16. The personal care product according to claim 1, wherein the first ingredient is an active antiperspirant salt, the second ingredient is an activity enhancement agent and the predetermined effect is enhanced wetness protection.

17. The personal care product according to claim 16, wherein the active antiperspirant salt comprises a material selected from the group consisting of aluminum zirconium pentachlorohydrex GLY, aluminum zirconium trichlorohydrex GLY, aluminum chlorohydrate, and mixtures thereof, and the activity enhancement agent comprises a material selected from the group consisting of calcium chloride, glycine, and mixtures thereof.

18. The personal care product according to claim 16, wherein the first portion comprises the active antiperspirant salt in an amount of from about 10 to about 50 weight percent (USP), and the second portion comprises the activity enhancement agent in an amount of from about 0.3 to about 12 weight percent.

19. The personal care product according to claim 1, wherein one of the first and second portions has a solid emulsion composition and the other of the first and second portions has a solid wax composition.

20. A method for manufacturing an antiperspirant product, the method comprising the steps of:
depositing a first portion of the antiperspirant product into a mold and allowing the first portion to at least partially solidify, the first portion comprising a first ingredient; and
depositing a second portion of the antiperspirant product into the mold and allowing the second portion to at least partially solidify, the second portion having a composition different from the first portion and comprising a second ingredient effective to interact with the first ingredient to produce a predetermined effect.
